(19) United States
(12) Patent Application Publication
(10) Pub. No.: US 2026/0284265 A1
(43) Pub. Date: Sep. 24, 2026

(54) METHOD FOR THE PREPARATION OF AN ADIPOSE TISSUE GRAFT

(71) Applicant: Paolo Gobbi Frattini s.r.l., Milano (IT)

(72) Inventor: Paolo Giuseppe GOBBI FRATTINI, Sondalo (IT)

(73) Assignee: Paolo Gobbi Frattini s.r.l., Milano (IT)

(21) Appl. No.: 19/471,856

(22) PCT Filed: Jan. 19, 2024
(86) PCT No.: PCT/EP2024/051304

§ 371 Date: Oct. 2, 2025

(30) Foreign Application Priority Data
Apr. 5, 2023 (IT) 102023000006693

(51) Int. Cl. A61L 27/36; A61J 1/10

(52) U.S. Cl. CPC A61L 27/3687; A61L 27/3604; A61L 27/3691; A61J 1/10

(57) ABSTRACT

Method for preparing a dehydrated adipose cells graft starting from unprocessed adipose cells, the method comprising the following successive steps:
a) introducing a portion of unprocessed adipose tissue into a device suitable for rinsing the adipose tissue;
b) rinsing the unprocessed adipose tissue using a sterile rinsing liquid;
c) removing the liquid from the device containing the adipose tissue, obtaining a dehydrated adipose cells graft;
wherein
the sterile rinsing liquid is supplemented with a mixture of oxygen-ozone thereby obtaining a liquid ozonized solution.

### BACKGROUND OF THE INVENTION

The present invention relates to a method for the preparation of human or animal adipose tissue grafts for allotransplantation or preferably for autologous transplant. In particular, the present invention relates to the treatment with ozone of the adipose tissue cells collected from the body of a human patient or an animal during the preparation of the graft.

In the following description, graft means a suitable quantity of adipose cells extracted from the body of an individual, treated, and that are ready to be implanted in the body of the same individual (autotransplantation, autologous transplant) or of a different individual (allotransplantation).

The transfer of adipose tissue from a point rich in adipose tissue like e.g. abdomen or thighs to a desired point like e.g. breasts or glutei is a procedure of reconstructive or aesthetic surgery known in the art. E.g., such a procedure can be used for reconstructing a breast after a surgical intervention for removing a tumour, or for a purely aesthetic intervention of additive mastoplasty. The use of adipose tissue coming from the same patient (autologous transplant) reduces the risk of transplant rejection and allows to prevent the use of artificial prosthesis (e.g. silicone prosthesis).

In the art, it is known getting the adipose tissue from a patient in order to prepare a graft through techniques like suction-assisted lipoplasty, ultrasound-assisted lipoplasty, excisional lipectomy, laser lipoplasty, water jet lipoplasty or the like.

In the art, it is known treating adipose tissue/cells so extracted from the body (not treated or unprocessed cells) in a sterile way, rinsing them with a sterile solution having the aim of removing impurities (see further) and liquids, with the aim of preparing a so-called dehydrated adipose tissue graft having a volume not too different from the volume that is intended to be placed in the site of grafting. In fact, the presence of liquids that are re-absorbed from the body after grafting can lead to poor aesthetic results.

It is important to note that in the adipose tissue/cells extracted from the body there are provided even cells different from adipose cells, in particular adipose-derived stem cells, endothelial progenitor cells, perivascular cells, macrophages, fibroblasts, lymphatic cells, which are connected to the adipose cells through the matrix of connective tissue. As known, stem cells are multipotent regenerative cells with the potential to differentiate into a plurality of other cell types performing one or more specific functions, and have the capacity of auto-regeneration.

The presence of such stem and regenerative cells is desired in the graft, in that they have a positive effect on the long-term success of grafting, e.g. promoting angiogenesis.

In the art, using sterile solutions for rinsing unprocessed adipose cells is known; such sterile rinsing solutions comprise sterile water, saline, Ringer's lactate, Ringer's acetate, phosphate-buffered saline, crystalloid solutions, colloidal solutions, 5% glucose in water, Hartmann's solution and the like.

Moreover, it is known that adipose tissue grafting can lead to undesired effects like infections, necrosis, reabsorption of the graft by the body, calcifications and scarring, which derive at least in part from the lack of neovascularization of the graft and from the necrosis of the grafted tissue.

One of the reasons that can lead to graft rejection is the presence of impurities in the graft, like e.g. red blood cells, white blood cells, and/or free lipids (oil). In fact, during the extraction of adipose cells, or of their successive processing e.g. through centrifugation, filtration, decantation or sedimentation, the membranes of such cells can be damaged leaking the content of the lipid droplets inside the adipose cells. It is known that such free lipids can lead to inflammation of the surrounding tissues, and in the worst cases, to necrosis and graft rejection.

Another cause of inflammation potentially leading to graft rejection is viral or bacterial contamination. Body tissues are normally sterile, but once the cells have been extracted from the body, they can be contaminated by virus and/or bacteria that can be airborne or present on surfaces.

In the art, there are known devices in the form of bags made of flexible material for preparing grafts of adipose tissues, that are described e.g. in EP2424463B1 describing a device known on the market under the name of Puregraft®, or in the document IT102015000064575 (UB20154901 A1). Such devices are sterile bags wherein the unprocessed adipose cells extracted from human body are inserted into a chamber provided with a filter having pores of suitable dimensions. In said chamber a rinsing liquid is introduced for removing impurities, allowing the removal of free lipids, white blood cells and red blood cells. The procedure is performed sterilely. The removal of the rinsing liquid allows to provide a preparation containing processed cells having a reduced liquid content, which constitute a dehydrated graft that can be grafted in the desired anatomical area immediately after collection and rinsing or of a dehydrated graft that can be cryopreserved in order to implant it in a subsequent moment.

The same document EP2424463B1 describes an alternative embodiment of the same invention, providing the use of a device comprising two chambers shown in the FIGS. 13, 14 of the document. Such chambers have substantially the shape of rigid cylinders in fluid communication, and from the point of view of performing the method, said cylinders can be likened to the chambers of the flexible bags.

Moreover, on the market there are available devices alternative to the above-described ones for separating adipose cells from other types of cells, impurities and liquids, wherein such separation is performed through decantation/sedimentation, or centrifugation, or a purification system through fragmentation with balls. A system for separating adipose cells through centrifugation is known under the commercial name Sepax® manufactured by Biosafe SA and described in the document EP1144026B1. A further system of separation of adipose cells is known under the commercial name Celution®, manufactured by Cytori Therapeutics and described in the document EP2380970B1. A system of separation of adipose cells through fragmentation with balls is known under the commercial name Lipogems®, manufactured by Lipogems and described in the document WO2015140737A1.

US 2017/224869 A1 discloses a method for producing a tissue product. The method comprises selecting an adipose tissue, treating the tissue to remove substantially all cellular material from the tissue, suspending the tissue in a liquid to form a suspension, layering the suspension in a mould, and freezing and drying the suspension to form a porous sponge.

CN 106492288 A discloses injectable acellular fat matrix particles prepared from adipose tissue by a method comprising the steps of providing animal fat tissue, rinsing, disinfecting, rinsing, repeated freezing and thawing, cutting into thin slices, then soaking in water, degreasing and rinsing.

A method known in the art for preparing an adipose tissue graft comprises the following steps:
a) Introducing a portion of unprocessed adipose tissue in a chamber of a device;
b) Rinsing the unprocessed adipose tissue using a sterile liquid;
c) Removing the liquid from the chamber containing said adipose cells, so obtaining a rinsed and dehydrated adipose tissue.

Ozone (O₃) is a gas naturally present in the atmosphere; its molecule consists of three atoms of oxygen. It is a highly unstable gas tending to rapidly decompose to molecular oxygen (O₂). It is a strong oxidizing agent. Ozone is soluble in water.

The therapeutic potential of ozone has been known for a long time, but its clinical application is limited by its strong cytotoxicity at high doses. E.g. there are known devices emitting ozone for sanitizing indoor air, due to its known bactericidal and virucidal activity.

It is known that the exposition to low concentrations of ozone induces the so-called eustress in cells, through the activation of anti-oxidant response pathways to stress, which are probably responsible for the therapeutic effect observed in the clinical practice.

### SUMMARY OF THE INVENTION

In view of the described state of the art, the aim of the present invention is providing a method and a device for performing such method capable of improving the success of adipose cell grafting.

According to the present invention, such aim is obtained through the method defined in the claims.

The present invention provides a method comprising the already known steps a), b), c), wherein the rinsing step b) is performed by placing said adipose cells in contact with a suitable quantity of ozone (O₃). Said ozone can be placed in contact with cells in the form of a liquid solution containing a suitable quantity of dissolved ozone (ozonized solution), or in the form of a mixture of oxygen/ozone gas in suitable quantities for cell fumigation. Preferably, the treatment with ozone is performed using a gaseous solution containing 0.1 to 8% of ozone (O₃) in in a gaseous solution containing 99.9 to 92% of oxygen (O₂). The quantity of ozone in oxygen can vary 0.1 to 80 micrograms per millilitre. Indicatively, the final ozonized solution contains 1 μg to 80 μg of ozone per mL of solution, equivalent to 0.1-8% of ozone in oxygen weight/volume. The bubbling time in ultra-filtered water, or in saline solution (0.9% NaCl) or in one of the sterile liquids used for rinsing, needed to form the ozonized solution is a time comprised in 0.1-10 minutes, preferably 0.1-4 minutes. The quantity/volume of sterile liquid to be ozonized can vary between 1 mL and 2000 mL; in case of big grafts of adipose tissues can reach 5000 mL.

In a preferred embodiment of the method according to the present invention said treatment with ozone occurs inside a device having the shape of a flexible bag shown in the Figures. Said bag comprises three chambers.

In a particularly preferred embodiment, the first chamber of said flexible bag is provided to the final user already filled with a sterile rinsing liquid chosen from the group consisting of sterile water, saline solution, Ringer's lactate, Ringer's acetate, phosphate-buffered saline, crystalloid solutions, colloidal solutions, 5% glucose in water solutions, Hartmann's solutions.

In a particularly preferred embodiment, said flexible bag is supported by a dedicated support allowing to keep said bag in a vertical position, with its longitudinal axis alternatively parallel or perpendicular to the ground, in order to perform more efficiently the different steps of the method.

Substantially, said support comprises a rectangular plate with a superficial face for supporting said flexible bag, an extension of one of the short sides and an extension of one of the long sides bent in the opposed direction with respect to the face supporting the flexible bag, and four hooks for connecting said flexible bag to said supporting face.

In alternative embodiments, the separation of adipose cells from other types of cells can be performed through decantation/sedimentation, or centrifugation, or fragmentation with balls. In other embodiments of the present invention, ozone is placed in contact with adipose cells using devices already on the market, which provide the use of containers different from flexible bags, generally rigid cylindrical containers. E.g., the devices described in EP1144026B1 (Sepax), EP2424463B1 (Puregraft), EP2380970B1 (Celution) e WO2015140737A1 (Lipogems) can be adapted in order to insert a rinsing step of adipose cells with a previously prepared ozonized solution.

The method of treatment with ozone according to the present invention is provided with two main advantages.

The first advantage is that ozone is provided with a known disinfecting activity, therefore is capable of destroying possible bacterial and/or viral contaminations present in the adipose cells, so preventing the occurrence of infections once the graft has been grafted in the body of a patient, and the potential rejection of the graft in the long-term.

The second advantage is that the treatment with low quantities of ozone can promote the anti-oxidant response of cells, activating adipogenesis and the stimulation of stem and regenerative cells contained in the graft of adipose tissue, so improving the probabilities of success of the grafting. Stem cells treated with ozone tend to be more numerous, vital and active, increasing their efficacy when they are grafted in the patient.

### DESCRIPTION OF THE DRAWINGS

Further advantages and properties of the present invention are disclosed in the following description, in which exemplary, non-limiting embodiments of the present invention are explained in detail based on the drawings:

FIG. 1 shows a schematic representation of a flexible bag device for preparing a cell graft according to a preferred embodiment, front view;

FIG. 2 shows said flexible bag device, in a longitudinal section according to the line A-A of FIG. 1;

FIG. 3 shows said flexible bag device, in a transversal section along the line B-B of FIG. 1;

FIG. 4 shows schematically the possibility of connecting a bag device with a syringe containing a sterile rinsing solution or alternatively an ozone generator;

FIG. 5 shows schematically the connection between an ozone generator and said flexible bag;

FIG. 6 shows schematically the connection between a syringe containing an ozonized solution or alternatively ozone and said flexible bag;

FIG. 7 consists of FIGS. 7A and 7B which show schematically the flexible bag device supported by a dedicated support. In particular, FIG. 7A shows the bag vertically supported by said support, with its longitudinal axis perpendicular to the ground in a front view, FIG. 7B shows the bag vertically supported by said support with its longitudinal axis parallel to the floor in a front view, FIG. 7C shows the bag vertically supported with its longitudinal axis perpendicular to the ground in a transparent axonometric view, while FIG. 7D shows the bag vertically supported by said support with its longitudinal axis parallel to the ground in a transparent axonometric view.

### SUMMARY OF THE INVENTION

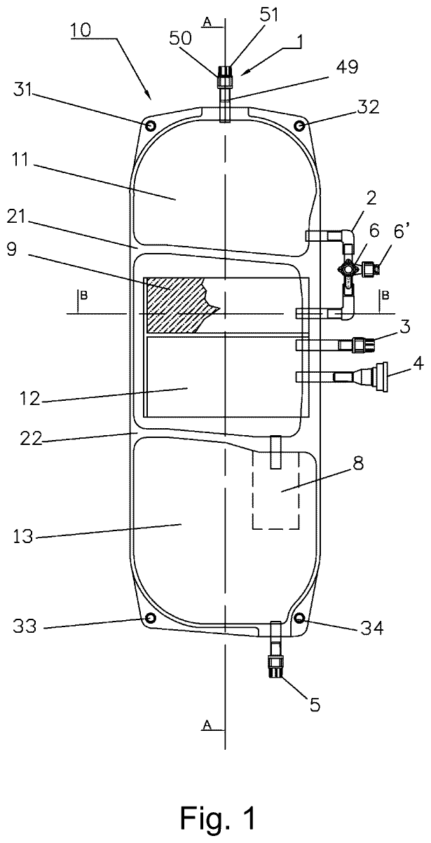

The device for preparing adipose tissue grafts that is exemplarily shown in FIGS. 1-3 is a bag 10 made of a flexible plastic material.

Said bag 10 is a closed system, allowing to sterilely process unprocessed adipose cells preventing their contact with environmental air; this allows to prevent bacterial and/or viral contaminations of the graft which could lead to infections and rejection of the graft.

Said bag 10 when empty has a flat shape, and in a top view is provided with a substantially rectangular shape, with two short sides and two long sides. The bag is manufactured by welding two equal flexible sheets of a suitable plastic material capable of being sterilized during production, and not degrading when placed in contact with ozone, according to the present invention. The material must also be capable of tolerating the temperatures needed for cryo-conserving the graft. Two welding 21, 22 divide the rectangular area of the bag in three hollow chambers 11, 12, 13.

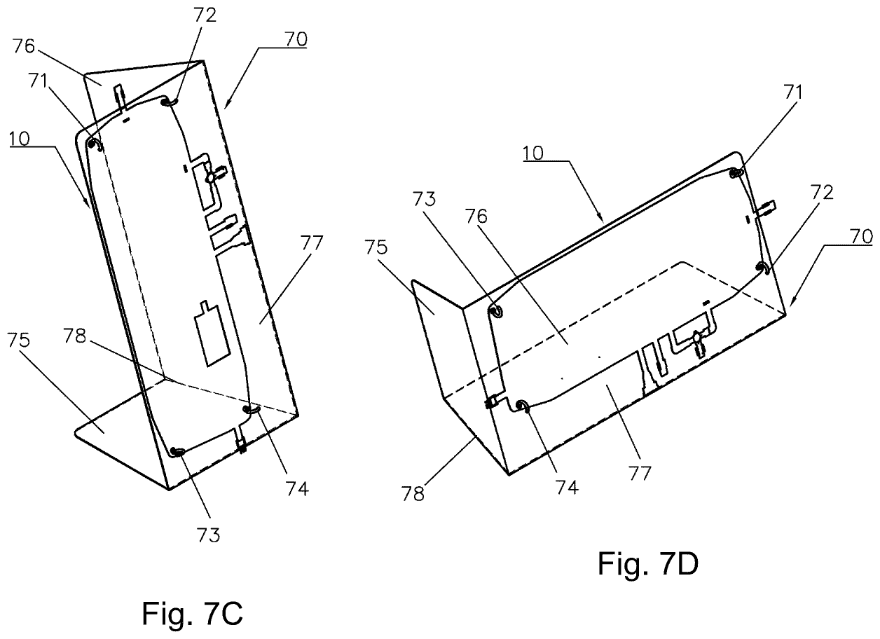

Said bag 10 is provided with four holes 31, 32, 33, 34 obtained near its angles, for the connection with a support device 70, shown in FIGS. 7A-7D.

The first hollow chamber 11 is provided for receiving and containing a sterile rinsing liquid (sterile water, saline, Ringer's lactate, Ringer's acetate, phosphate-buffered saline, crystalloid solutions, colloidal solutions, 5% glucose in water solutions, Hartmann's solutions, ozonized solutions or the like) that is inserted through an inlet port 1 comprising a loading flexible tube 49 provided with a hermetic sealing plug 50 that can be opened through an external axial pressure and automatically closed when said pressure stops. The sealing plug 50 is preferably of the kind described in EP2667839B1 and is provided with a threaded end 51.

The second hollow chamber 12 is provided for receiving the raw or unprocessed adipose tissue, wherein said tissue is rinsed and undergoes a treatment with a rinsing liquid and ozone (ozonized solution) according to the present invention, and successively dehydrated.

In a known way, said hollow chamber 12 comprises a separator filter 9 having the shape of a bag with substantially known features, which filter comprises pores of dimensions suitable for passing the impurities of the unprocessed adipose tissue, while holding adipose cells and stem and regenerative cells. E.g. said filter 9 can be made of polyester or PES with porosities of 80 or 105 μm, inside a porosity field ranging 50 to 200 μm, or of Nylon.

Said second chamber 12 further comprises an inlet port 4 on one of its long sides, provided for sterilely introducing, preferably through a syringe, an unprocessed adipose tissue into said chamber 12, and in particular into said filter 9, and an outlet port 3 on a long side provided for sterilely extracting, preferably through a syringe, the dehydrated graft of adipose tissue after the processing of said cells occurred insides said chamber 12.

Moreover, there is a tube 2 provided for connecting said first chamber 11 and said second chamber 12. On said tube 2 there is provided a known three-way valve 6, allowing to adjust the outflow of liquid from said first chamber 11 to said second chamber 12. Said three-way valve 6 is provided with an inlet port 6′ that can be used even for connecting said tube 2 with a (not shown) syringe with the aim of inserting active principles of different kinds (e.g. antibiotics or inhibitors of the immune system in case of allotransplantation) inside said second chamber 12. The inlet port 6′ is provided with a sealing plug of the same kind of the above-mentioned plug 50.

The third draining hollow chamber 13 is provided for draining impurities and liquids coming from the second chamber 12. The third chamber 13 comprises an outlet port 5 allowing to drain the liquid from the third chamber 13, and eventually from said bag 10.

The connection between said second chamber 12 and said third draining chamber 13 occurs through a non-return valve 8 of known type (schematized only in FIG. 1), allowing the passage of liquids from the second chamber 12 to the third chamber 13. Said valve 8 is normally closed and allows the passage of liquids in just one direction, preventing the rise of the draining liquid from said third chamber 13 to said second chamber 12. The passage of the draining liquid occurs through the pressure exerted by the liquid on said valve 8. In other words, as soon as the liquid is pushed from the second chamber 12, it ends directly in the third chamber 13, and it is not possible to hold it in the second chamber 12.

In an alternative embodiment (not shown here, but visible in the patent IT102015000064575), the connection between second chamber 12 and third chamber 13 is realized analogously to the connection between first chamber 11 and second chamber 12, using a tube on which there is provided a tap or a known hose clamp. In this way, preventing the outflow of the rinsing liquid from the second chamber 12 to the third chamber 13 through the device closing said tube, the ozonized solution can be kept inside said second chamber 12 for a desired time, and the closing device can be opened when the necessary contact time between ozone and cells contained in said second chamber 12 has elapsed.

The inlet port 4 and the outlet port 3 for cells, as well as the inlet port 1 and the outlet port 5 for liquids, are preferably provided for coupling with a syringe of standard needleless type, e.g. with a connection of Luer Lock type, well known in the art.

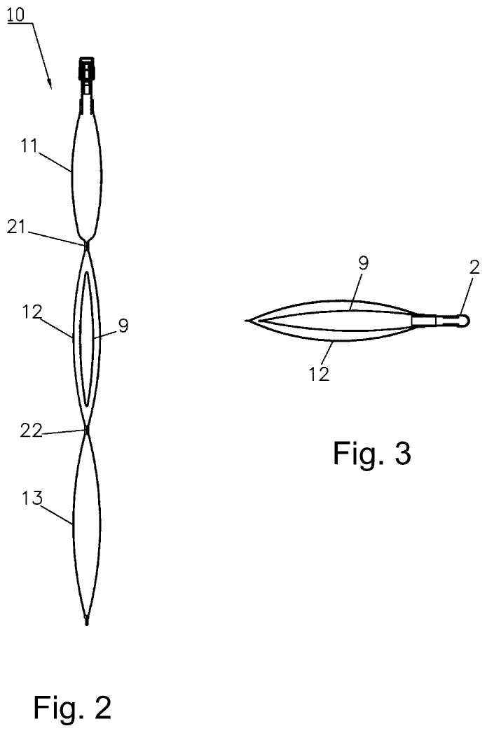

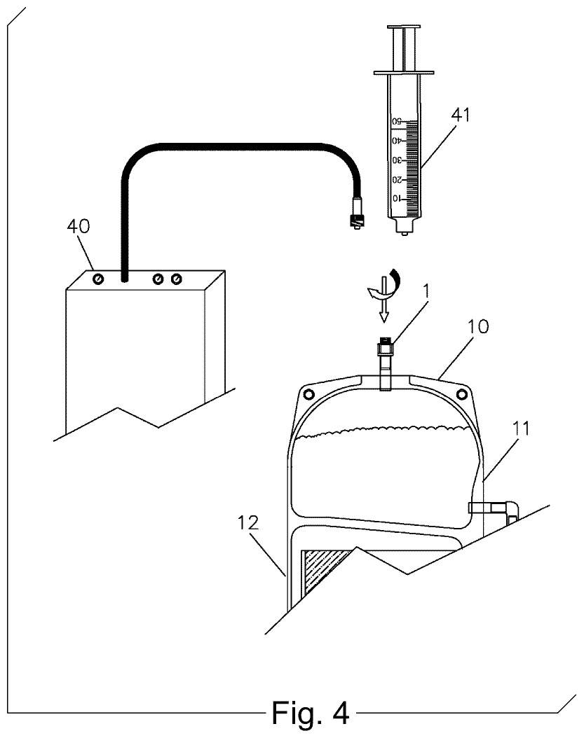

The longitudinal section of the flexible bag 10 (FIG. 2) allows to better observe the filter 9 inside the second chamber 12, while the transversal section (FIG. 3) allows to detect the relationship among chamber 12, filter 9 and tube 2. In fact, the filter 9 is inside the chamber 12, and is provided with three points of access, i.e. the inlet of the tube 2, the inlet port 4 and the outlet port 3 for cells.

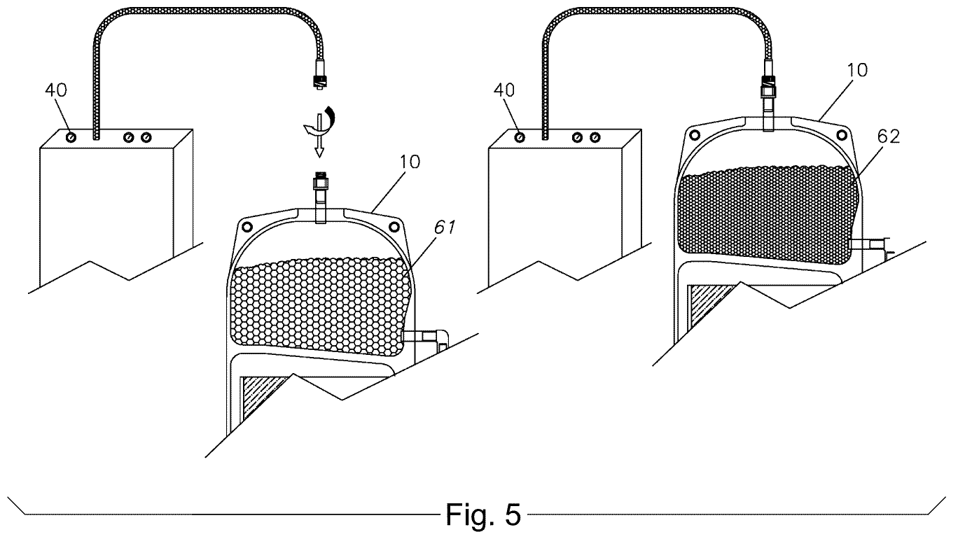

The inlet port 1 of the first chamber 11 is used alternatively for filling the first chamber 11 with a sterile rinsing solution contained in a syringe 41, or for connecting said chamber to a generator 40 of an oxygen-ozone mixture (see FIG. 4).

In a preferred embodiment, the first chamber 11 of the flexible bag 10 contains about 250 mL of liquid, while the chamber 13 can contain about 350 mL. Anyway, it is apparent that the flexible bag 10 can be manufactured in different dimensions, adapting the dimensions of its chambers 11, 12, 13 to operational needs, e.g. manufacturing a flexible bag suitable for preparing grafts of large quantities of adipose tissue, with ensuing increase of the dimensions of the chambers.

Now the performing of the method according to the present invention will be described in its preferred embodiment, i.e. through the use of a bag 10 as shown in FIGS. 1-3.

A human operator introduces a quantity of unprocessed adipose tissue (raw cells) collected from the body of a patient into the second chamber 12, in particular inside the filter 9, using a needleless syringe that is coupled to the inlet port 4. The three-way valve 6 is adjusted so as not to allow the passage of substances between the chambers 11 and 12.

Successively, the operator introduces the sterile rinsing solution in the first chamber 11 through its specific inlet port 1.

The operator places the bag 10 vertically, e.g. using the support 70, shown in FIGS. 7A-7D. The three-way valve 6 is opened, so as to allow the passage due to gravity of the sterile rinsing solution from the first chamber 11 to the second chamber 12.

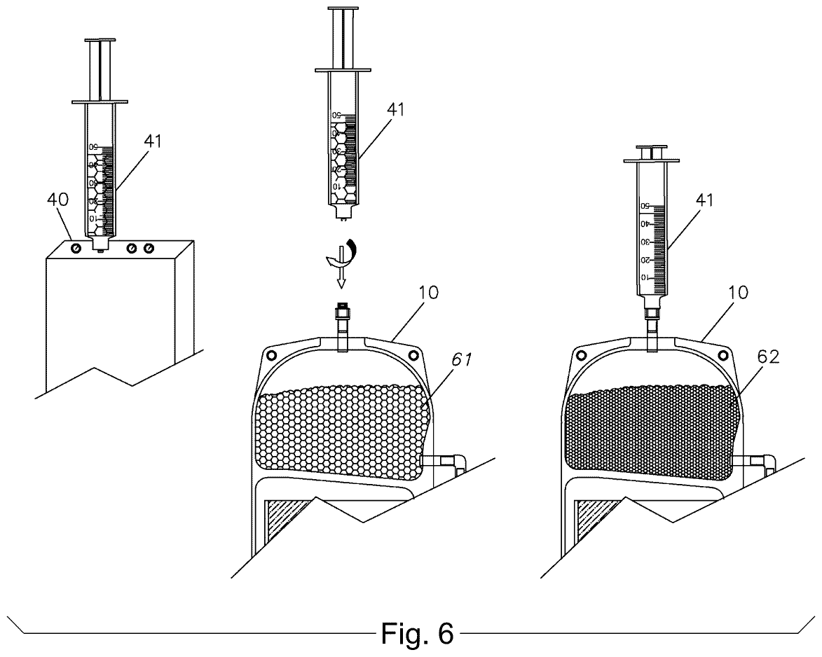

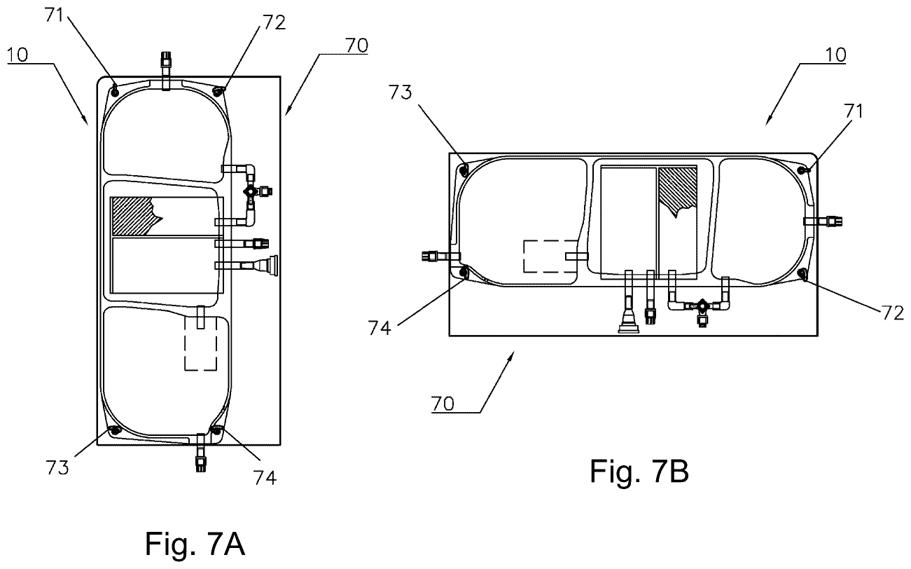

It is worth precising that the contact of the cells in the second chamber 12 with ozone can occur in different ways:
When said three-way valve 6 is closed, in the first chamber 11 is inserted, or it is already present, a known quantity of sterile liquid solution through the inlet port 1. The inlet port 1 is successively connected with a generator 40 of a mixture of oxygen-ozone, bubbling the mixture oxygen-ozone in the liquid 61 for a time suitable for obtaining the desired final concentration of ozone, i.e. the ozonized solution 62 (FIG. 5). Alternatively, a known quantity of oxygen-ozone mixture is taken with a syringe 41 and inserted in the first chamber 11 already containing a known quantity of sterile rinsing solution 61 (FIG. 6). Once obtained the ozonized rinsing solution, by opening the three-way valve 6, the solution is channelled into the second chamber 12, and therefore ozone comes into contact with the cells held inside the filter 9.
In the first chamber 11 a previously prepared liquid solution is inserted, containing a suitable quantity of ozone (ozonized solution), that is then channelled from the first chamber 11 to the second chamber 12 wherein the cells are contained through the first tube 2.
Inside the second chamber 12 containing cells and a defined quantity of liquid, a suitable quantity of oxygen-ozone mixture is inserted, using the entry port 6′ of the second chamber 12. In this case, too, the ozone, by mixing with the liquid wherein cells are immersed, forms an ozonized solution.

The desired contact time is maintained, then the ozonized liquid is channelled from the second chamber 12 to the third chamber 13.

In a preferred embodiment, the flexible bag 10 is provided to the final user with the first chamber 11 already filled with a sterile rinsing solution chosen from the group consisting in sterile water, saline, Ringer's lactate, Ringer's acetate, phosphate-buffered saline, crystalloid solutions, colloidal solutions, 5% glucose in water solutions, Hartmann's solutions. In this way, the human operator only needs to insert into the filter 9 of the second chamber 12 the unprocessed adipose tissue and a suitable quantity of ozone in the first chamber 11.

In the flexible bag of standard dimensions, in the first chamber 11 there are provided 250 mL of sterile rinsing solution.

By skipping the step of filling the first chamber 11 the sterility of operation is improved and operations are simplified, in that the sterile rinsing liquid is pre-dosed, and therefore dosing the desired quantity of ozone to be inserted in the first chamber 11 for preparing the ozonized rinsing solution becomes simpler.

Once all the rinsing sterile ozonized solution passed from the first chamber 11 to the second chamber 12, the three-way valve 6 is closed and the operator agitates and squeezes said second chamber 12, so as to delicately mix the sterile rinsing solution and the adipose cells contained in said chamber 12. Through the separator filter 9 placed inside the second chamber 12, the action of the operator promotes the separation of impurities (white blood cells, red blood cells, where applicable free lipids) from the adipose cells and the stem and regenerative cells. This also allows to discard the excess liquids, that are drained to the third chamber 13, so obtaining a dehydrated graft, having a volume smaller than the volume of adipose tissue/cells originally collected. In the embodiment shown in FIGS. 1-3 provided with the non-return valve 8, said draining occurs as soon as the rinsing liquid enters into said second chamber 12.

In an alternative embodiment, not shown here but visible in the patent IT102015000064575, wherein the connection between the second chamber 12 and the third chamber 13 occurs through a tube provided with a device capable of blocking the flow of the liquid, the ozonized solution can be held for a defined time inside said second chamber 12, and therefore the contact time between cells and ozone can be controlled in a more precise way.

In a particularly preferred embodiment shown in FIG. 7, the bag 10 is supported by a dedicated support 70, described in detail in the following, allowing to maintain its longitudinal axis perpendicular to the ground during the steps a) introduction of adipose tissue into the filter 9 and b) rinsing of the adipose tissue with the ozonized solution. In the step c) removal of the processed adipose tissue from the chamber 12, said support 70 is laid down on the long side of the bag 10 provided with the cell outlet port 3, so as to facilitate the outflow of all the cells from the filter 9. In said particularly preferred embodiment, the outflow of processed adipose cells is performed by pressing said chamber 12 with a spatula (not shown).

Obviously, the above-described rinsing operation can be performed one or more times, opportunely manoeuvring the three-way valve 6 and the inlet port of the first chamber 11. Optionally two or more rinsing steps of the cells can be performed, optionally using alternatively a rinsing solution according to the known art and an ozonized solution according to the present invention.

FIG. 4 schematically shows an embodiment wherein a syringe 41, preferably a Luer Lock syringe, is connected to the inlet port 1 of the chamber 11 for inserting a defined quantity of sterile rinsing liquid, with the three-way valve 6 in its closed condition, so that said rinsing liquid remains inside the first chamber 11. Thereafter, the syringe 41 is removed and the same inlet port 1 is used for supplying said first chamber 11 with a defined quantity of an oxygen-ozone mixture coming from the ozone generator 40. The inlet port 1 is then closed, and this point in the chamber 11 there is provided an ozonized solution that is channelled due to gravity into the chamber 12 by manoeuvring the three-way valve 6.

Inside said second chamber 12 the contact between ozonized solution and adipose cells is maintained for a suitable time, and then the ozonized solution is channelled into the third chamber 13. If the flexible bag 10 according to FIGS. 1-3 is used, that is not provided with a device preventing the outflow of the ozonized solution from the second chamber 12 to the third chamber 13, the suitable contact time between ozone and cells is obtained through using a suitable quantity of rinsing liquid, that flows on the cells for the time required for obtaining the effect of ozone.

At the end of rinsing operations, the removal of the processed and dehydrated adipose cells is performed, by extracting said cells, i.e. the dehydrated graft, from the outlet port 3 provided in the second chamber 12, preferably through a syringe.

The draining liquid flowed in the third chamber 13 is extracted from the bag 10 through the outlet port 5, which is provided with a hermetically closing plug 50 that can be opened through an external axial pressure and can close automatically when said pressure is stopped. The closing plug is preferably of the type described in patent EP2667839B1.

FIGS. 7A-7D show the bag 10 supported during its use by a three-dimensional support 70.

Said support 70 consists in a rectangular plate 77 made of rigid material, metal or a plastic material, provided with two perpendicular extensions 75 and 76 respectively, of a short and a long side of said plate 77, which are bent in opposed directions with respect to a superficial face of said rectangular plate 77 that is intended for supporting said bag 10. The two extensions 75 and 76 meet at a common edge 78.

Said support 70 comprises four hooks 71, 72, 73, 74 for connecting said bag 10, in particular for connecting its corresponding holes 31, 32, 33, 34 obtained in the corners of said bag 10, to said support 70.

Said in another words, said three-dimensional support 70 comprises:
a first rectangular-shaped surface 77 on which the back face of the bag 10 leans;
a second surface 75 having the shape of a scalene trapezoid bent to an angle of about 90° with respect to said first surface 77, that allows to said support 70 to support the bag 10 nearly vertically with respect to the ground, in a first position wherein the longitudinal axis A-A is perpendicular with respect to the ground;
a third surface 76 having the shape of an isosceles trapezium bent to an angle of about 90° with respect to said first surface 77, that allows to said support 70 to support the bag 10 nearly vertically with respect to the ground, in a second position wherein the transversal axis B-B is perpendicular with respect to the ground.

In the alternative embodiments not providing the use of a flexible bag, a sterile pre-ozonized solution can be used, or a suitable quantity of gaseous oxygen-ozone mixture can be inserted for fumigating the cells contained in rigid chambers, which gaseous mixture combining with the solutions contained in said chambers produces an ozonized solution.

1. Method for preparing a dehydrated adipose cells graft starting from unprocessed adipose cells, the method comprising the following successive steps:
introducing a portion of unprocessed adipose tissue into a device suitable for rinsing the adipose tissue;
b) rinsing the unprocessed adipose tissue using a sterile rinsing liquid;
c) removing the sterile rinsing liquid from the device containing the adipose tissue, obtaining a dehydrated adipose cells graft;
wherein
the sterile rinsing liquid is supplemented with a mixture of oxygen-ozone, thereby obtaining a sterile rinsing liquid ozonized solution.

2. The method for preparing a dehydrated adipose cells graft starting from unprocessed adipose cells according to claim 1, using a gaseous solution comprising 0.1-8% of ozone and 99.9 to 92% of oxygen.

3. The method for preparing a dehydrated adipose cells graft starting from unprocessed adipose cells according to claim 1, wherein the sterile rinsing liquid ozonized solution placed into contact with the adipose cells contains a concentration of ozone ranging 1 μg to 80 μg of ozone per mL.

4. The method for preparing a dehydrated adipose cells graft starting from unprocessed adipose cells according to claim 1, wherein the sterile rinsing liquid ozonized solution is placed into contact with the adipose cells for a contact time ranging 0.1-10 minutes.

5. The method for preparing a dehydrated adipose cells graft starting from unprocessed adipose cells according to claim 4, wherein the sterile rinsing liquid ozonized solution is generated by insufflating the mixture of oxygen-ozone inside a chamber containing a sterile rinsing liquid, and containing the adipose cells to be treated.

6. A device for preparing a dehydrated adipose cells graft starting from unprocessed adipose cells according to the method defined in claim 1, the device being a sterile flexible bag provided with two short sides and two long sides, comprising:
a first chamber, provided with an entry port for receiving and containing a sterile rinsing liquid;
a second chamber, provided with a filter for housing the non-processed adipose tissue, capable of retaining adipose and staminal and regenerative cells, wherein the adipose cells are rinsed and treated with ozone, and subsequently dehydrated;
a third draining chamber, for draining liquids and adipose cells coming from the second chamber;
a tube placed on one of the long sides of the bag, for connecting and putting in communication the first chamber and the second chamber, the tube being provided with a control element;
an entry port, placed on one of the long sides of the bag in an area the second chamber, to allow the sterile introduction of the non-processed adipose tissue into the second chamber;
an exit port, placed on one of the long sides the bag in an area of the second chamber, to allow the sterile extraction of the dehydrated adipose tissue graft after treatment of adipose cells from the second chamber;
a non-return valve for connecting and putting in communication the second chamber and the third chamber.

7. The device for preparing a dehydrated adipose cells graft starting from unprocessed adipose cells according to claim 6, wherein the entry port for introducing the sterile rinsing liquid is provided with a hermetic closing plug openable through external axial pressure and automatically closable at the cessation of the axial pressure.

8. The device for preparing a dehydrated adipose cells graft starting from unprocessed adipose cells according to claim 6, provided to a final user with the first chamber pre-filled with a sterile rinsing liquid.

9. A supporting device for preparing a dehydrated graft of adipose cells starting from unprocessed adipose cells according to claim 6, wherein the bag is provided with a supporting device, allowing to keep the bag in a vertical position, with its longitudinal axis alternatively perpendicular or parallel with respect to the ground, the supporting device comprising:
a first rectangular surface on which a posterior face of the bag is supported;
a second surface that is an extension of a short side of the first rectangular surface, bent in a direction opposed to the first rectangular surface of the bag;
a third surface that is the extension of a long side of the first rectangular surface, bent in a direction opposed to the first rectangular surface supporting the bag;
four hooks located on the first rectangular surface for connecting the first rectangular surface to four holes located at four corners of the sterile flexible bag.

10. The supporting device for preparing a dehydrated adipose cells graft starting from unprocessed adipose cells according to claim 9, wherein the supporting device keeps the bag with its longitudinal axis perpendicular to the ground during the step a) introducing a portion of unprocessed adipose tissue into the second chamber and step b) rinsing the unprocessed adipose tissue, while it is laid down so that the longitudinal axis of the bag is parallel to the ground during the step c) and removing the treated adipose tissue from the second chamber.

11. The method for preparing a dehydrated adipose cells graft starting from unprocessed adipose cells using a device according to claim 6, wherein the contact between ozone and adipose cells contained in the second chamber occurs by inserting into the first chamber a sterile rinsing liquid and then a gaseous oxygen-ozone mixture forming the sterile rinsing liquid ozonized solution, and successively flowing the ozonized solution into the second chamber through the tube and the control element, and finally draining the ozonized-solution into the third chamber through the non-return valve or alternatively through a tube provided with a control element.

12. The method for preparing a dehydrated adipose cells graft starting from unprocessed adipose cells using a device according to claim 6, wherein contact between ozone and adipose cells contained in the second chamber occurs by inserting into the first chamber a previously ozonized liquid solution containing a suitable quantity of ozone, that is successively flowed from the first chamber to the second chamber wherein adipose cells are housed, through the tube; the ozonized solution is kept in contact with the adipose cells for a desired time, and successively is drained into the third chamber through the non-return valve or alternatively through a tube provided with a control element.

13. The method for preparing a dehydrated adipose cells graft starting from unprocessed adipose cells using a device according to claim 6, wherein the contact between ozone and adipose cells contained in the second chamber occurs by inserting into the second chamber a suitable quantity of oxygen-ozone mixture using the entry port of a three-way valve and maintaining gas in the second chamber for a desired contact time, successively draining the ozonized solution formed in the second chamber from the second chamber to the third chamber through the non-return valve or alternatively through a tube provided with a control element.

14. The method for preparing a dehydrated adipose cells graft starting from unprocessed adipose cells according to claim 1, wherein separation of adipose cells from other types of cells is performed by centrifugation, decantation or sedimentation, or stirring through beads, inserting inside rigid containers provided for the performing of the separation methods an ozonized solution or a gaseous mixture of oxygen-ozone for fumigation.
